(19) United States
(12) Patent Application Publication
(10) Pub. No.: US 2024/0423869 A1
(43) Pub. Date: Dec. 26, 2024

(54) AUGMENTED REALITY SYSTEM FOR CARDIOPULMONARY RESUSCITATION

(71) Applicant: THE JOHNS HOPKINS UNIVERSITY, Baltimore, MD (US)

(72) Inventors: Keith KLEINMAN, Baltimore, MD (US); Therese L. CANARES, Baltimore, MD (US); James DEAN, Rockville, MD (US); Justin JEFFERS, Baltimore, MD (US); Blake SCHREURS, Columbia, MD (US); Brandon SCOTT, Las Vegas, NV (US)

(21) Appl. No.: 18/687,539

(22) PCT Filed: Sep. 7, 2022
(86) PCT No.: PCT/US2022/042686

§ 371 Date: Feb. 28, 2024

Related U.S. Application Data
(60) Provisional application No. 63/241,622, filed on Sep. 8, 2021.

(51) Int. Cl. A61H 31/00; G16H 20/30; G16H 70/20

(52) U.S. Cl. CPC A61H 31/005; A61H 31/007; G16H 20/30; G16H 70/20; A61H 2201/10; A61H 2201/1253; A61H 2201/1619; A61H 2201/5012; A61H 2201/5015; A61H 2201/5043; A61H 2201/5069; A61H 2201/5084; A61H 2201/5097; A61H 2203/0456; A61H 2230/201; A61H 2230/305; A61H 2230/405

(57) ABSTRACT

A system for facilitating cardiopulmonary resuscitation (CPR) by a CPR performer on a patient includes first, second, and third sensors configured to be positioned at least partially between a hand of the CPR performer and a chest of the patient. The first sensor is configured to measure a depth of compressions provided by the CPR performer on the patient. The second sensor is configured to measure a rate of the compressions. The third sensor is configured to measure a recoil of the compressions. The system also includes a computing system configured to receive data from the first, second, and third sensors. The computing system is also configured to compare the received data to stored data in a library that corresponds to the received data. The computing system is also configured to generate one or more outputs in response to the comparison.

### CROSS-REFERENCE TO RELATED APPLICATIONS

This application is the national stage entry of International Patent Application No. PCT/US2022/042686, filed on Sep. 7, 2022, and published as WO 2023/038921 A1 on Mar. 16, 2023, which claims the benefit of U.S. Provisional Patent Application Ser. No. 63/241,622, filed on Sep. 8, 2021, which are hereby incorporated by reference herein in their entireties.

### FIELD OF THE DISCLOSURE

The present disclosure relates generally to cardiopulmonary resuscitation (CPR). More particularly, the present disclosure relates to an augmented reality (AR) system to facilitate CPR.

### BACKGROUND OF THE DISCLOSURE

While pediatric cardiac arrest is rare, when it does occur, the fatality rate is high (e.g., >96%). This may be because providers use poor CPR techniques or deviate from resuscitation guidelines. Proper CPR on children is difficult because the depth and hand placement of the chest compressions varies based upon the age and/or size of the child. Conventional CPR systems have flaws with visibility and usability. Therefore, what is needed is a system and method to improve CPR that is more visible and usable.

### SUMMARY

In accordance with an aspect of the present disclosure, a system for facilitating cardiopulmonary resuscitation (CPR) by a CPR performer on a patient is disclosed. The system includes a first sensor configured to be positioned at least partially between a hand of the CPR performer and a chest of the patient. The first sensor is configured to measure a depth of compressions performed by the CPR performer on the patient. The system also includes a second sensor configured to be positioned at least partially between the hand of the CPR performer and the chest of the patient. The second sensor is configured to measure a rate of the compressions. The system also includes a third sensor configured to be positioned at least partially between the hand of the CPR performer and the chest of the patient. The third sensor is configured to measure a recoil of the compressions. The system also includes a computing system configured to receive data from the first sensor, the second sensor, and the third sensor. The computing system is configured to compare the received data to stored data in a library that corresponds to the received data. The computing system is also configured to generate one or more outputs in response to the comparison.

In another embodiment, the system includes a plurality of sensors. The sensors include a first sensor configured to be positioned at least partially between a hand of the CPR performer and a chest of the patient. The first sensor is configured to measure a depth of compressions. The sensors also include a second sensor configured to be positioned at least partially between the hand of the CPR performer and the chest of the patient. The second sensor is configured to measure a rate of the compressions. The sensors also include a third sensor configured to be positioned at least partially between the hand of the CPR performer and the chest of the patient. The third sensor is configured to measure a recoil of the compressions. The sensors include a fourth sensor configured to be positioned at least partially between the hand of the CPR performer and the chest of the patient. The fourth sensor is configured to measure a location of a heart of the patient. The sensors include a fifth sensor configured to be positioned proximate to a mouth of the patient. The fifth sensor is configured to measure a level of carbon dioxide that is released at an end of an exhaled breath. The system also includes a computing system configured to receive data from the sensors. The computing system is also configured to compare the data to a library. The library includes stored data that corresponds to the data received from the sensors. The stored data includes pediatric CPR guidelines provided by the American Heart Association (AHA) or another medical governing body. The computing system is also configured to generate one or more outputs in response to the comparison. The one or more outputs instruct the CPR performer how to modify the CPR on the patient to reduce differences between the received data and the stored data. The system also includes an augmented reality (AR) headset configured to be positioned on a head of the CPR performer and to display the one or more outputs. The one or more outputs include a measured depth indicator corresponding to the depth of the compressions measured by the first sensor. The one or more outputs also include a measured rate indicator corresponding to the rate of the compressions measured by the second sensor. The one or more outputs also include a predetermined depth range based upon the stored data. The one or more outputs also include a predetermined rate range based upon the stored data. The predetermined depth range and the predetermined rate range form a box. The measured depth indicator and the measured rate indicator being inside the box indicate that the CPR performed by the CPR performer is satisfactory. One or both of the measured depth indicator and the measured rate indicator being outside the box indicate that the CPR performed by the CPR performer is unsatisfactory.

A method for facilitating cardiopulmonary resuscitation (CPR) by a CPR performer on a patient is also disclosed. The method includes measuring a depth of compressions performed by the CPR performer on the patient using a first sensor. The method also includes measuring a rate of the compressions performed by the CPR performer on the patient using a second sensor. The method also includes measuring a recoil of the compressions performed by the CPR performer on the patient using a third sensor. The first, second, and third sensors are positioned at least partially between a hand of the CPR performer and a chest of the patient. The method also includes comparing the measured depth, rate, and recoil to a stored depth, rate, and recoil that are stored in a library. The method also includes generating one or more outputs in response to the comparison.

### BRIEF DESCRIPTION OF THE FIGURES

FIG. 1 illustrates a perspective view of a CPR performer performing CPR on a patient, according to an embodiment.

FIG. 2 illustrates a schematic view of a system for facilitating CPR, according to an embodiment of the present disclosure.

FIG. 3 illustrates a flowchart of a method for facilitating CPR, according to an embodiment of the present disclosure.

FIGS. 4A and 4B illustrate images that may be output by the system and/or method, according to an embodiment of the present disclosure.

FIGS. 5A-5D illustrate additional images that may be output by the system and/or method, according to an embodiment of the present disclosure.

FIG. 6 illustrates a perspective view of a measurement device, according to an embodiment

FIG. 7 illustrates a compression testing setup, according to an embodiment.

### DETAILED DESCRIPTION OF THE PREFERRED EMBODIMENTS

The presently disclosed subject matter now will be described more fully hereinafter with reference to the accompanying Drawings, in which some, but not all embodiments of the disclosures are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

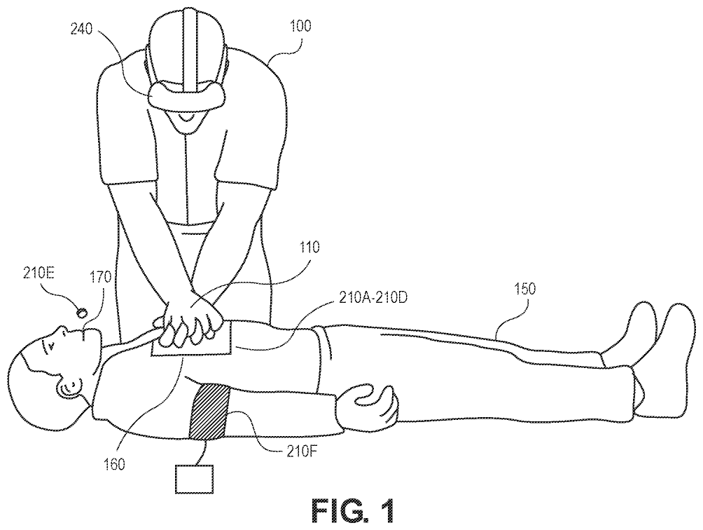

FIG. 1 illustrates a perspective view of a CPR performer 100 performing CPR on a patient 150, according to an embodiment. The CPR performer 100 may be or include a healthcare professional (e.g., a doctor, a nurse, pre-hospital EMS worker, etc.). In other embodiments, the CPR performer 100 may not be a healthcare professional. For example, the CPR performer 100 may be a person with no CPR training.

The patient 150 may require CPR for a variety of reasons, for example, because the heart stopped due to arrythmia (e.g., blunt chest trauma, electrolyte abnormality, previously known or unknown pre-existing condition, etc.), lack of oxygen (e.g., suffocation, respiratory failure, drowning, etc.), trauma, shock, or the like. The patient 150 may be an adult or a child. For example, the patient 150 may be a child that is younger than 15 years old, younger than 10 years old, younger than 5 years old, younger than 3 years old, or younger than 1 year old. In another example, the patient 150 may be less than about 100 pounds, less than about 80 pounds, less than about 60 pounds, less than about 40 pounds, less than about 20 pounds, less than about 10 pounds, or less than about 5 pounds. In another example, the patient 150 may be less than 6 feet tall, less than 5 feet tall, less than 4 feet tall, less than 3 feet tall, less than 2 feet tall/long, or less than 1 foot tall/long.

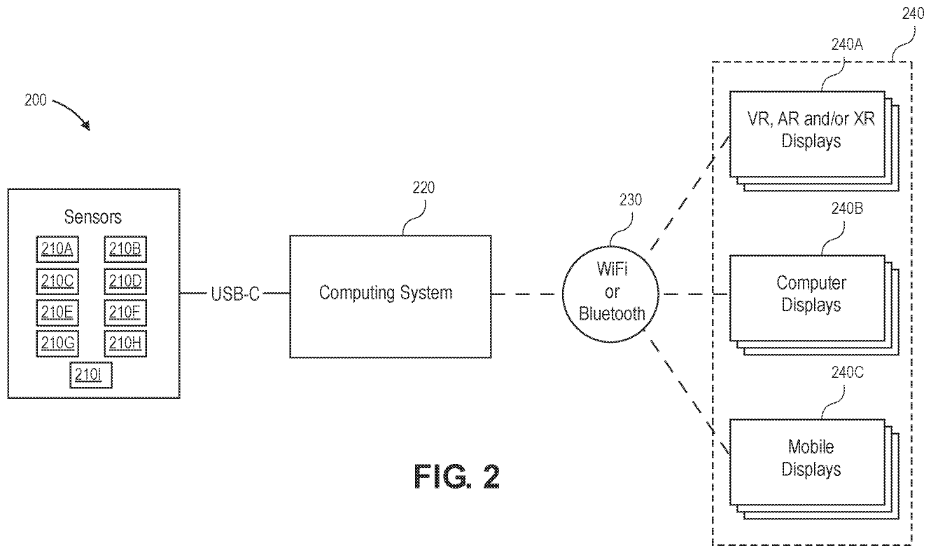

FIG. 2 illustrates a schematic view of a system 200 for facilitating CPR by the CPR performer 100 on the patient 150, according to an embodiment of the present disclosure. Referring now to FIGS. 1 and 2, the system 200 includes one or more sensors (nine are shown: 210A-210I). The sensors 210A-210I may be used in any combination, and one or more of the sensors 210A-210I may be omitted.

The first sensor 210A may be positioned at least partially between the CPR performer 100 and the patient 150. More particularly, the first sensor 210A may be positioned at least partially between the hand(s) 110 of the CPR performer 100 and the chest 160 of the patient 150. For example, the first sensor 210A may be coupled to the hand(s) 110 of the CPR performer 100, to defibrillator pads being used by the CPR performer 100, to the chest 160 of the patient 150, or a combination thereof. The defibrillator pads may be used with or without internal CPR feedback monitoring sensors. The first sensor 210A may be configured to measure the depth of the CPR compressions. The first sensor 210A may be or include an accelerometer, a gyroscope, a magnetometer, or a combination thereof.

The second sensor 210B may be positioned at least partially between the CPR performer 100 and the patient 150. More particularly, the second sensor 210B may be positioned at least partially between the hand(s) 110 of the CPR performer 100 and the chest 160 of the patient 150. For example, the second sensor 210B may be coupled to the hand(s) 110 of the CPR performer 100, to defibrillator pads being used by the CPR performer 100, to the chest 160 of the patient 150, or a combination thereof. The defibrillator pads may be used with or without internal CPR feedback monitoring sensors. The second sensor 210B may be configured to measure the rate of the CPR compressions (e.g., in compressions per minute). The second sensor 210B may be or include an accelerometer, a gyroscope, a magnetometer, or a combination thereof.

The third sensor 210C may be positioned at least partially between the CPR performer 100 and the patient 150. More particularly, the third sensor 210C may be positioned at least partially between the hand(s) 110 of the CPR performer 100 and the chest 160 of the patient 150. For example, the third sensor 210C may be coupled to the hand(s) 110 of the CPR performer 100, to defibrillator pads being used by the CPR performer 100, to the chest 160 of the patient 150, or a combination thereof. The defibrillator pads may be used with or without internal CPR feedback monitoring sensors. The third sensor 210C may be configured to measure the recoil of the CPR compressions. The third sensor 210C may be or include an accelerometer, a gyroscope, a magnetometer, or a combination thereof.

A fourth sensor 210D may also be positioned at least partially between the CPR performer 100 and the patient 150. More particularly, the fourth sensor 210D may be positioned at least partially between the hand(s) 110 of the CPR performer 100 and the chest 160 of the patient 150. For example, the fourth sensor 210D may be coupled to the hand(s) 110 of the CPR performer 100, to defibrillator pads being used by the CPR performer 100, to the chest 160 of the patient 150, or a combination thereof. In one embodiment, the sensors 210A-210D may be combined into a single sensor. The fourth sensor 210D may be configured to measure the location of the hand(s) 110 of the CPR performer 100, the location of the sensors 210A-210D, the location of one or more internal members of the patient 150 (e.g., the heart, ribs, sternum, etc.), or a combination thereof. In one embodiment, the sensor 210D may be configured to measure/determine the location of the patient's heart and/or to measure/determine the location of the hand(s) 160 of the CPR performer 100 with respect to the location of the patient's heart. In another embodiment, the sensor 210D may be configured to measure the number of fingers and/or hands being used to perform the CPR. For example, the sensor 210D may be configured to measure whether two fingers are being used (e.g., for babies), one hand is being used (e.g., for small children), or two hands are being used (e.g., for big kids and/or adults).

A fifth sensor 210E may be positioned proximate to the mouth 170 of the patient 150. The sensor 210E may be configured to measure the gas flowing out of the mouth 170 of the patient 150 during the CPR. More particularly, the sensor 210E may be configured to measure the level of carbon dioxide that is released at the end of an exhaled breath (i.e., end tidal CO2).

A sixth sensor 210F may be positioned at least partially around and/or on the patient 150. For example, the sensor 210F may be or include a blood pressure cuff that is configured to be positioned around the arm or wrist of the patient 150 and to measure the blood pressure of the patient 150.

A seventh sensor 210G may be positioned on the wrist, hand, finger, ankle, foot, toe, and/or ear of the patient 150. The sensor 210G may be or include a pulse oximetry (SpO2) sensor that is configured to measure the oxygenation of blood in a specific moment of time (e.g., the oxygen saturation).

An eighth sensor 210H may be positioned at least partially in the patient's artery and be configured to provide invasive blood pressure monitoring. More particularly, the sensor 210H may include a small catheter that is placed in the artery and connected to a calibrated pressure transducer that reports the systolic, diastolic, and/or mean arterial pressure.

One or more ninth sensors 210I may be positioned on the patient's forehead and/or abdomen. The sensor(s) 210I may be near infrared spectroscopy (NIRS) sensors that are configured to measure the oxygenation of tissues. The sensor on the forehead may be a surrogate for brain oxygenation/perfusion, and the sensor on the abdomen may be a surrogate for gut oxygenation/perfusion.

The system 200 may also include a computing system 220. The computing system 220 may be configured to receive the data from the sensors 210A-210I either through one or more wires or wirelessly. For example, the computing system 220 may be connected to the sensors 210A-210I via a USB-C cable, a category 5 ethernet cable, BLUETOOTH®, or the like. The computing system 220 may be configured to process the data and to produce one or more outputs, as described below.

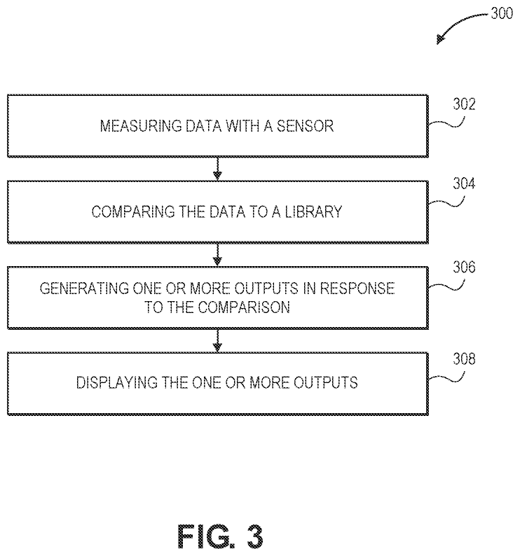

The computing system 220 may include one or more processors and a memory system. The memory system may include one or more non-transitory computer-readable media storing instructions that, when executed by at least one of the one or more processors, cause the computing system to perform operations. Illustrative operations are described below with reference to the flowchart in FIG. 3.

The system 200 may also include a wireless internet hub (e.g., a Wi-Fi router and/or BLUETOOTH® device) 230. The hub 230 may be configured to wirelessly transmit the output(s) from the computing system 220.

The system 200 may also include one or more displays 240 (e.g., 240A-240C). Multiple displays 240A-240C may be used so that more than one person may be able to view the outputs. One or more first displays 240A may be or include virtual reality (VR), augmented reality (AR), extended reality (XR), and/or mixed reality headset(s) (e.g., glasses), which may be worn on the head of the CPR performer 100 or others in the area. AR is a modality that can visually display real-time feedback (e.g., the outputs) to the CPR performer 100 on the CPR performer's chest compression technique. One or more second displays 240B may be or include computer monitors (e.g., desktop monitors, laptop monitors, etc.). One or more third displays 240C may be or include mobile displays (e.g., tablets, smartphones, etc.).

FIG. 3 illustrates a flowchart of a method 300 for facilitating CPR, according to an embodiment of the present disclosure. The method 300 may be performed by the system 200 while the CPR performer 100 performs CPR on the patient 150. An illustrative order of the method 300 is provided below; however, one or more steps of the method 300 may be performed in a different order, combined, split into sub-steps, repeated, or omitted. The method 300 may begin once the sensors 210A-210I are positioned, and the CPR performer 100 begins performing CPR on the patient 150.

The method 300 may include measuring data with the sensors 210A-210I, as at 302. As described above, the data may be or include the depth of the compressions by the CPR performer 100 from the first sensor 210A. The data may also or instead include the rate of the compressions by the CPR performer 100 from the second sensor 210B. The data may also or instead include the chest recoil of the patient 150 from the third sensor 210C. The data may also or instead include the location of the hand(s) and/or the number of fingers/hands on the chest 160 of the patient 150 from the fourth sensor 210D. The data may also or instead include the end tidal CO2 from the fifth sensor 210E. The data may also or instead include the blood pressure of the patient 150 from the sixth sensor 210F. The data may also or instead include the blood oxygenation from the seventh sensor 210G. The data may also or instead include the invasive blood pressure data from the eighth sensor 210H. The data may also or instead include the tissue oxygenation data from the ninth sensor 210I. The data may then be received by the computing system 220 from the sensors 210A-210I.

The method 300 may also include comparing the data to a library, as at 304. This may include comparing the received data (from 302) to stored data that is stored in a library in the computing system 220. The stored data may be provided by the American Heart Association (AHA) or another medical governing body. In one embodiment, the stored data may be for pediatric (e.g., infant) patients.

The stored data may correspond to the received data. For example, the stored data may include a predetermined range of depths of the compressions (e.g., for infants), a predetermined range of rates of the compressions (e.g., for infants), a predetermined range of locations of the hand(s) 110 and/or finger(s) of the CPR performer 100 on the chest 160 of the patient 150 (e.g., for infants), a predetermined range of end tidal CO2 (e.g., for infants), a predetermined range of the blood pressure (e.g., for infants), or a combination thereof. Thus, in one example, the depth of the compressions from the sensor 210A may be compared to the predetermined range of depths of compressions in the library, the rate of the compressions from the sensor 210B may be compared to the predetermined range of rates of the compressions in the library, the chest recoil from the sensor 210C may be compared the to predetermined range of chest recoil in the library, the location of the hand(s) 160 from the sensor 210D may be compared to the predetermined range of locations in the library, the end tidal CO2 from the sensor 210E may be compared to the predetermined range of end tidal CO2 in the library, the blood pressure from the sensor 210F may be compared to the predetermined range of blood pressure in the library, or a combination thereof. In one embodiment, the library may have different sets of stored data based upon the age, height, and/or weight of the patient 150, and the data from the sensors 210A-210I may be compared against the set of stored data that corresponds to the age, height, and/or weight of the patient 150.

The method 300 may also include generating one or more outputs in response to comparing the data to the library, as at 306. In one embodiment, the outputs may help to instruct the CPR performer 100 how to modify his/her CPR to more closely follow the CPR guidelines for infants based upon the differences between the measured/received data and the stored data.

The method 300 may also include displaying the one or more outputs, as at 308. In one embodiment, the output may be images that may be displayed using the displays 240A-240C.

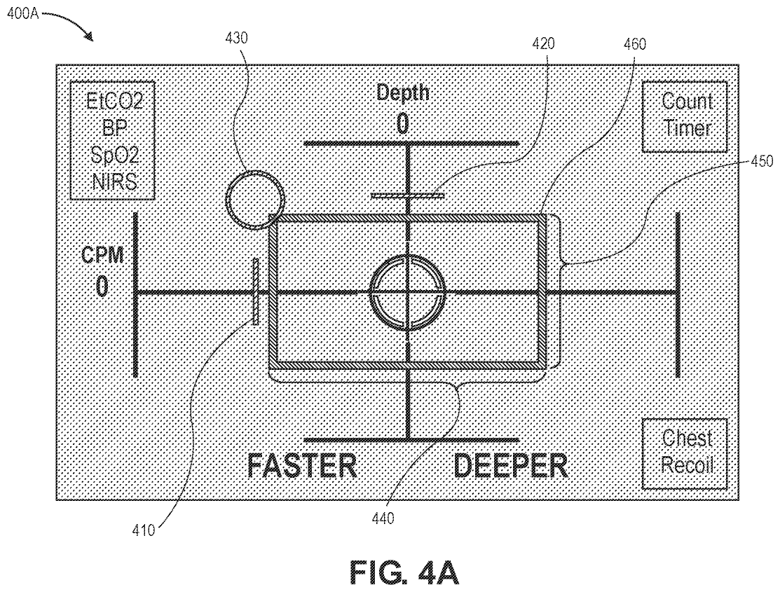

FIGS. 4A and 4B illustrate images 400A, 400B that may be output (e.g., displayed) by the system 200, according to an embodiment. The images 400A, 400B may include an X axis (horizontal axis) and a Y axis (vertical axis). In the examples shown, the X axis represents the depth of the compressions, and the Y axis represents the rate of the compressions. The images 400A, 400B may also include a measured depth indicator 410 that indicates the depth of the compressions measured by the first sensor 210A along the X axis. The images 400A, 400B may also include a measured rate indicator 420 that indicates the rate of the compressions measured by the second sensor 210B along the Y axis. The images 400A, 400B may also include a measured combined indicator 430 at the intersection of the indicators 410, 420. The measured combined indicator 430 may be or include a circle, a cross (e.g., cross-hairs), or the like.

The images 400A, 400B may also include a predetermined depth range 440, which may be obtained from the library. The images 400A, 400B may also include predetermined rate range 450, which may be obtained from the library. As may be seen, the predetermined depth range 440 and the predetermined rate range 450 may form a box 460. As will be appreciated, the CPR performer 100 may modify his/her depth and/or rate in response to viewing the displays 240A-240C to try to get his/her measured combined indicator 430 within the box 460. The images 400A, 400B may also include commands. For example, the first image 400A is instructing the CPR performer 100 to make the compressions faster and deeper, and the second image 400B is instructing the CPR performer 100 that the current compressions are too fast and too deep (i.e., slow down and go more shallow).

The images 400A, 400B may also include other outputs from the sensors 210A-210I such as the chest recoil, the EtCO2, the blood pressure, the SpO2, the NIRS, and the count timer. The chest recoil may be feedback if the CPR performer 100 allows the chest to fully recoil in between compressions. This allows the heart to refill with blood. The countdown timer may count down from a predetermined time (e.g., 10 seconds) whenever a pause is detected to minimize interruptions in the CPR.

In another embodiment, the output may also or instead be pulses sent to the sensor(s) 210A-210I. For example, the computing system 220 may provide a metronome type functionality to provide a reference rate for performing CPR at a predetermined pace (e.g., based upon the age, height, weight, etc. of the patient 150). Thus, the pulses may be transmitted at the metronome rate to the sensors 210A-210D, which may transmit the pulses to the hand(s) 110 of the CPR performer 100 in the form of electrical pulses and/or vibrations. In one example, stronger pulses may instruct the CPR performer 100 to make deeper compressions, and lighter pulses may instruct the CPR performer 100 to make shallower compressions, or vice versa. In another example, faster pulses may instruct the CPR performer 100 to increase the rate of the compressions, and slower pulses may instruct the CPR performer 100 to decrease the rate of the compressions, or vice versa. In yet another example, pulses on an upper portion of the sensor(s) 210A-210D may instruct the CPR performer 100 to move his/her hands up (e.g., toward the patient's head), pulses on a lower portion of the sensor(s) 210A-210D may instruct the CPR performer 100 to move his/her hands down (e.g., toward the patient's feet), pulses on a side portion of the sensor(s) 210A-210D may instruct the CPR performer 100 to move his/her hands to that side.

In another embodiment, the output may also or instead be audible. For example, the output may be or include voice instructions provided to the CPR performer 100 to vary or maintain the rate of the compressions, the depth of the compressions, etc.

### Additional Embodiments

Each year, 20,000 children in the United States suffer a cardiac arrest, but only 17%-50% survive. Survival is associated with delivery of high-quality chest compressions. Despite the importance of high-quality chest compressions, healthcare providers adhere to the rate and depth guidelines recommended by the Pediatric Advanced Life Support (PALS) program for chest compression only about 20%-40% of the time. Major causes of insufficient compression quality in children include variation in size and chest wall compliance, in addition to the emotional stress of a dying child. Rates of high-quality chest compressions improve by about 32% when a quality cardiopulmonary resuscitation (qCPR) coach (e.g., a teammate giving real-time feedback on chest compression performance) is present. While effective, qCPR coaching is resource and personnel intensive, limiting its use in prehospital or community settings, and diminishing the quality of care based on location.

The system 200 and method 300 described herein may provide real-time CPR feedback without additional personnel. As described above, they may include an augmented reality (AR) feedback system (e.g., headset 240A) called AR-CPR that provides visual feedback in a medical provider's field of view, thus improving the quality of chest compressions in real-time. The preliminary data, gathered from 34 subjects, demonstrates the feasibility of a head-mounted display 240A to offer instantaneous visual feedback on CPR quality. More particularly, the system 200 and method 300 improved the performance of chest compression rate and depth, raising guideline-compliant CPR from 17% to 73%.

One long-term goal is to improve pediatric survival from cardiac arrest. More particularly, an objective of the present disclosure is to refine AR-CPR for accuracy and precision of rate and depth, enhance usability, add recoil sensors (e.g., sensor 240C), and test AR-CPR in a large-scale high-fidelity simulated setting. These refinements may improve PALS guideline adherence and the rate of high-quality CPR that is performed, positioning AR-CPR as the most effective and user-friendly pediatric CPR feedback system available.

### Iterative Refinement and Small-Scale Testing of AR-CPR

The system 200 and method 300 have upgraded the rate and depth sensors 210A, 210B for AR-CPR using an upgraded inertial measurement unit (IMU) to measure chest compressions accurately and precisely in real-time. The system 200 and method 300 also use enhanced AR-CPR's hardware with direct wireless communication between the rate and depth sensors 210A, 210B and the AR-CPR display 240, allowing for an always-on system capable of instantaneous use. The system 200 and method 300 also detect chest recoil through real-time data analysis of the upgraded IMU system, adding another component of chest compression feedback. The accuracy and precision of the AR-CPR rate, depth, and recoil sensors 240A-240C may be determined by comparing the measurements to known accurate and precise measurement systems in a simulated setting.

### International Multicenter Simulation-Based Evaluation of AR-CPR

The AR-CPR's ability to guide pediatric chest compressions may be determined by comparing the rate of high-quality compressions over predetermined (e.g., 1-minute) intervals while using AR-CPR to compressions performed to using the standard quality CPR coaches. This determination may use a randomized, multicenter, simulation-based, non-inferiority study design in collaboration with a simulation research collaboration.

The usability of AR-CPR's software may be iteratively evaluated with a mixed-methods approach, measured with the system usability scale and feedback from semi-structured interviews, to inform further iterative change to the functionality and design of the system 200. This may help to refine the AR-CPR to improve pediatric chest compression quality in a simulated setting.

### High Quality CPR is a Temporizing Measure to Keep Blood Circulating

High quality chest compressions are performed to replace the function of the beating heart during a cardiac arrest (e.g., when a heart stops beating). When correctly done, the compression of the chest forces the blood from the heart to the vital organs. Chest compressions only provide 20%-30% of the cardiac output, or volume of blood flowing out of the heart per unit time, compared to a naturally beating heart. However, the goal is to maintain optimal circulation until the original the heart can be restarted. Thus, CPR can prevent irreversible brain and other organ damage by minimizing “no-flow” and “low-flow” time. To effectively provide sufficient blood flow, the CPR performer 100 may compress at a rate of 100-120 compressions per minute (CPM), at a depth of 4-6 cm depending on the child's age, and allow the chest to fully recoil to enable the heart to refill with blood.

The CPR performer 100 may also maximize the chest compression fraction (CCF), the proportion of time that chest compressions are provided to the patient 150 in cardiac arrest, place his/her hands in the correct location over the heart, and avoid excessive ventilation. Attaining this high mark in all metrics simultaneously, and sustaining it, is uncommon, with adherence to rate and depth guidelines remaining around 20%-40%. Chest compression rate and depth are amongst the most difficult elements of CPR to do correctly. Cardiac output is dependent on the heart/compression rate and stroke volume (i.e., the volume of blood ejected from the heart with each beat/compression). Compressing too slowly can dangerously suppress cardiac rate. Compressing too quickly does not allow for sufficient time for the heart to refill with blood between each compression, which limits stroke volume. Both extremes limit cardiac output. The depth of each chest compression determines the degree of cardiac ventricular compression. Compressions that are too shallow result in insufficient stroke volume. Compressions that are too deep can result in unnecessary intrathoracic trauma and diminished stroke volume due to decreased ventricular filling time. Any of these errors in isolation or combined result in a “low-flow” state, asphyxiating the vital organs.

### Augmented Reality (AR)

The system 200 and method 300 described herein provide pediatric CPR feedback using augmented reality (e.g., an AR headset 240A). Augmented reality harnesses advances in microprocessors and display technology to show useful information directly in a user's field of view. It is a non-immersive tool that enhances a user's world with an overlay of information in real-time, while maintaining visualization of the user's actual physical environment. This is distinct from virtual reality (VR), which is designed to transport a user to a completely immersive virtual environment, minimizing all aspects of the user's surrounding physical environment. AR's subtle, non-immersive integration of information layered on top of the physical environment enables seamless and unobtrusive use in simulation and clinical settings.

### Augmented Reality Can Provide Useful Feedback to Assist in CPR

A healthcare provider (e.g., CPR performer 100) can wear AR glasses 240A, called AR-CPR, designed to recognize how deep and fast the CPR performer 100 is compressing, and determine the degree of chest recoil. The glasses 240A may provide positive reinforcement when the CPR performer 100 is within the AHA PALS recommended ranges and alert the CPR performer 100 when they are not, providing guidance towards high-quality CPR. If a community ED provider cares for a child in cardiac arrest, the instantaneous cognitive offloading and guidance about CPR may drastically improve the quality of care delivered. This change allows the remaining limited members of the resuscitation team to focus on performing other critical roles.

### AR-CPR is an Improvement Over Other Applications of Augmented Reality for CPR

AR-CPR provides specific quantitative and immediately actionable feedback to the CPR performer 100. Augmented reality feedback can help coordinate other interventions that occur during cardiac arrest, thereby streamlining their timing and further supporting the use of AR in cardiac arrest management.

### AR-CPR Components and User Interface

The system 200 that detects the occurrence and magnitude of each chest compression may include an inertial measurement unit (IMU), which may be part of one or more of the sensors 210A-210C. The IMU may be or include an accelerometer and/or gyroscope. Data measured by the IMU may then be transmitted via a cable (e.g., a USB-C cable) to the computing system 220 (FIG. 2) running an AR-CPR data analysis software that synthesizes the compression data. The data is may then be (e.g., wirelessly) transmitted to an AR head mounted display 240A that is running AR-CPR coaching software.

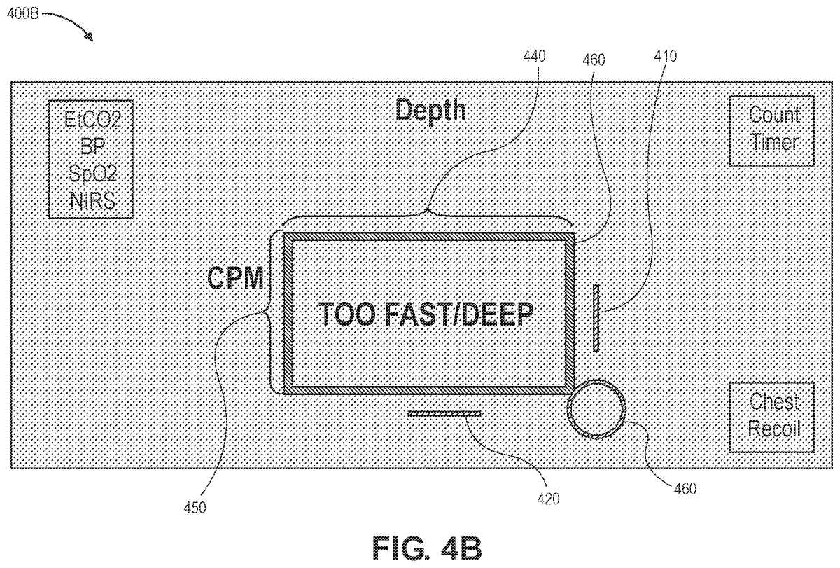

FIGS. 5A-5D illustrate additional images that may be output by the system 200 and/or method 300, according to an embodiment of the present disclosure. If the AR-CPR detects no compressions for a predetermined amount of time (e.g., three seconds), it may display “START COMPRESSIONS,” as shown in FIG. 5A. In-range compressions may be displayed with (e.g., green) markers within the larger green target box 460 and display of the word “GOOD,” as shown in FIG. 5B. The circle 430 may integrate both rate and depth information. Depth is displayed with a vertical marker 410 in centimeters (cm) with deeper depths displayed with a line lower to the bottom of the display. Rate is displayed with a horizontal marker 420 in compressions per minute (CPM), with faster rates displayed with a line farther to the right of the display.

Compressions that are detected outside of the goal rate or depth may result in the appropriate marker changing from green to red, the marker's 430 movement outside of the target box 460, and a text box displaying the nature of the user's deficit, as depicted by the “TOO FAST prompt, as shown in FIG. 5C. If the CPR performer 100 continues to compress sub-optimally with any metric out of range for greater than 3 seconds, a larger visual prompt displays guided feedback to correct the compressions, as depicted by the “GO SLOWER” prompt in FIG. 5D. The AR-CPR IMU may detect the chest compression impulses and calculate the rate and depth of each compression, when placed beneath the hand of the CPR performer 100.

### AR-CPR Prototype Testing—Initial Testing

Initial prototype testing in 34 Pediatric Emergency Department (PED) providers in a quaternary care children's hospital with simulated pediatric cardiac arrest scenarios demonstrated that AR-CPR improves chest compression adherence to AHA PALS guidelines from 17% (SD 26%) to 73% (SD 18%) as measured by AR-CPR (P<0.001), when comparing both rate and depth simultaneously. This unblinded randomized crossover study demonstrated that AR-CPR can change the CPR performer's behavior in real-time when used while performing chest compressions. Thematic analysis of exit interview transcripts from these subjects focused on usability of the headset 240A, anticipated barriers to AR-CPR use in clinical environments, and emotional response to chest compression performance.

Further prototype testing was performed with 34 General Emergency Department non-pediatric specialized nurses and clinical technicians from two community hospitals, using simulated pediatric cardiac arrest scenarios. These results similarly demonstrated that AR-CPR improves chest compression performance from 18% (SD 30%) adherence to AHA PALS guidelines to 87% (SD 12%) as measured by AR-CPR (P<0.001), when comparing both rate and depth simultaneously.

The AR-CPR may be refined to measure and deliver accurate and precise chest compression rate and depth feedback and incorporate chest recoil feedback. This functionality may be validated, sequentially analyzing the equipment to simulate real-world performance. An interclass correlation coefficient (ICC) of 0.95 may be used as an acceptable correlation to declare equivalence when comparing AR-CPR measurements to validated standards. A large-scale multicenter international non-inferiority simulation-based study may also be conducted that compares AR-CPR to qCPR coaching.

Refine AR-CPR to Improve the Quality of Chest Compression Feedback in Accordance with PALS Guidelines and Enhance the Hardware Usability

As mentioned above, the inertial measurement unit (IMU) may measure the rate and/or depth of the chest compressions accurately and precisely in real-time. The AR-CPR's hardware may include direct wireless communication between the sensors 210G-210I and the AR-CPR display 240, allowing for an always-on system capable of instantaneous use. The system 200 may also include chest recoil detection through real-time data analysis of the IMU, adding a relevant component of chest compression feedback. For example, the sensor 210C may be configured to measure the chest recoil.

### Upgrade AR-CPR Rate and Depth Sensors

To guide high quality chest compressions in clinical practice, the compression depth and rate measurements must be accurate and precise. As discussed above, the system 200 may include IMU (e.g., sensors 210A-210C), including a (e.g., triaxial) accelerometer, gyroscope, and/or a geomagnetic sensor to measure the acceleration, direction, magnitude, and time of an impulse. The computing system 220 can then calculate the depth of compression based upon these measurements. The computing system 220 may include sensor fusion software capable of (e.g., absolute) orientation vectoring. For example, the computing system 220 may combine the information from the three sensors (e.g., accelerometer, gyroscope, and/or a geomagnetic sensor) into one accurate vector signal, has comparatively low output noise, and automatic real-time calibration. This IMU is capable of accurately and precisely determining the occurrence and range of a chest compression, without the deficits of conventional IMUs, due to the fusion software and more advanced sensors, paving the way for clinical use.

### Alternatives

Position estimation error accumulation is a known challenge of all IMUs. To mitigate this, the system 200 and method 300 may include a dedicated drift characterization to determine what corrections (e.g., filtering) may improve accuracy.

### Boost AR-CPRs Usability by Engineering Direct Wireless Communication

The computing system 220 of the AR-CPR may be or include a notebook computer connected (e.g., via USB-C) to the IMU and a microcomputer. The AR-CPR coach application processes real-time measured compression data, collecting hundreds of datapoints per second. The immediate analysis of these measurements may then be performed on the notebook computer. This system has functioned as intended for prototype testing. The intended use of AR-CPR is to support lifesaving chest compressions when a child's heart is not beating. Thus, even delays of seconds can be important. In an embodiment, the AV headset 240A may directly communicate with the sensors 210A-210I, eliminating the notebook computer entirely. In an embodiment microcomputer may detect and analyze compression data in real-time and send chest compression feedback signals to the AV headset 240A programmed to listen for the signal, without the need for a notebook computer. This streamlined and more ergonomic system may be always-on, without any boot-time, and in a “Stand-By” state, allowing for instantaneous use when needed.

### Develop Chest Recoil Detection

For chest compressions to be effective, sufficient time and ventricular volume are needed for the heart to refill with blood between each chest compression. Chest recoil data may facilitate high-quality CPR, as it allows for sufficient ventricular volume filling time. If sufficient recoil is not provided, the heart is maintained in a constant state of partial compression, restricting the left ventricular volume, and increasing pressure. Thus, poor recoil prevents the heart from passively refilling with oxygen rich blood prior to the next compression, dangerously suppressing cardiac output. To correct this, The AR-CPR's sensor 210C may provide real-time chest recoil monitoring and feedback to the user. To do this, AR-CPR software may be updated to record both the depth of compressing the chest and the subsequent negative deflection that occurs with releasing the compression. If the chest is allowed to recoil by at least 95% of the initial linked compressive deflection, full recoil is reported to the user. Any negative (recoil) deflection of the IMU that is less than 95% of the initial linked compressive deflection may be reported as insufficient chest recoil. The recoil data may be integrated into the current user interface and be presented in the user's field of view. In an alternative embodiment, the system 200 may also include a force sensor to measure the change in compressive force directly, and the computing system 220 may extrapolate the recoil based at least partially upon the compressive force.

### Quantitative Analysis of Accuracy and Precision

The system 200 and method 300 may assess the accuracy and precision of the AR-CPR rate, depth, and/or recoil sensors 210A-210C by comparing its measurements to known accurate and precise measurement systems in a simulated setting.

### Single Axis Accuracy and Precision Testing

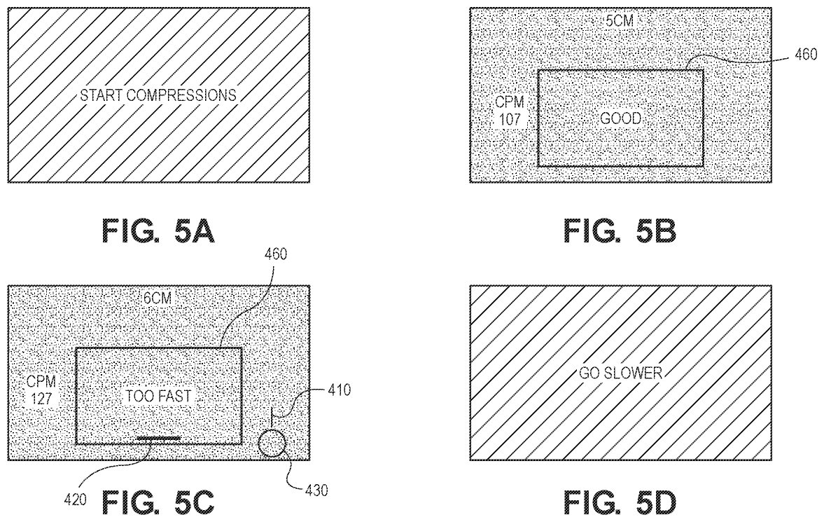

FIG. 6 illustrates a perspective view of a measurement device 600, according to an embodiment. The device 600 may be designed to travel a predetermined distance (e.g., five centimeters) in the y-axis by rotating a lever 610. The device 600 may be motorized for repeatability, providing a depth gauge to be used as a reference when measuring electronic sensors. The AR-CPR IMU may be placed on a slide 620 and repeatedly travel exactly five cm for a predetermined time (e.g., 18 minutes) to mimic the duration of the compressions. This may also ensure that AR-CPR is able to perform accurately and precisely over a prolonged duration. The recorded average distance moved, as detected by the AR-CPR sensors 210A-210I may be compared to the known constant movement of the slide 620 to determine that AR-CPR is accurate and precise in this standardized single axis movement environment. This process may be repeated with individual measurement slides built to travel four cm and six cm to determine that AR-CPR measures accurately and precisely across the full spectrum of goal depths for pediatric CPR as per AHA PALS.

### Comparison to a Demonstrated Accurate and Precise Compression System

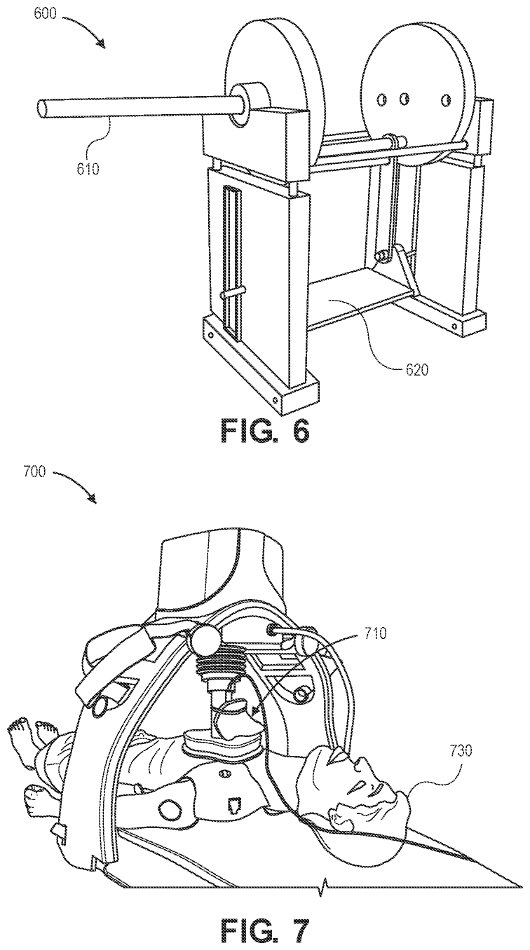

FIG. 7 illustrates a compression testing setup 700, according to an embodiment. The testing setup 700 may closely mimic the acceleration, angulation, and/or magnitude of the forces produced during chest compressions performed by humans, while maintaining a highly precise degree of consistency in compression rate and depth. The compression testing setup 700 is an FDA approved automated chest compression system for adult populations. The accuracy and precision of the compression rate of 102+/−2 compressions per minute and a depth of 5.3 cm+/−0.2 cm per compression has been well established. The compression testing setup 700 can therefore be used as a control to test the rate and depth accuracy and precision of AR-CPR.

As a secondary control, a first tracking system (e.g., an HTC Vive Tracker 3.0) 710 may also or instead be used in conjunction with a second tracking system (e.g., an HTC Vive Steam VR Tracking 2.0 system). The second tracking system may emit multiple sync pulses and laser lines. This allows tracking of the timings between pulses and lines to map the location, orientation, and/or velocity of each first tracker 710 to within a fraction of a millimeter in a 3-D space. This technology is called 3-D optical tracking localization. The second tracking system may also be tested with a mechanical reference instrument to determine the hardware and software in the first tracking system 710 in order to provide data in a manner consistent with required fidelity.

In a fully 3-D mapped environment, the first tracking system 710 may be mounted to a piston of the testing setup 700 which may measure the distance traveled and/or rate of impulse. This in turn, can be accurately calculated into a compression depth and rate. In this fully mapped environment, the testing system 700 may be activated on a simulation manikin 730 designed to approximate the chest wall mechanics of an actual child. The AR-CPR sensors 210A-210D may be placed between the chest wall and a compression arm of the testing setup 700, allowing it to detect the rate and/or depth of the chest compressions performed by the testing setup 700. The output of the AR-CPR system 200 and method 300 may be directly compared to the output of the second tracking system and to that of the testing setup's known compression metrics. In this model, the testing setup 700 may provide an accurate and precise chest compression rate and/or depth, allowing for validity assessment of the accuracy and precision of the system 200 and/or the first tracking system 710 in measuring compression rate and depth. A predetermined ICC (e.g., 0.95) may be used to declare equivalence between the system 200, the testing setup 700, the first tracking system 710, or a combination thereof.

### Measure Accuracy and Precision in Simulated Environments

A small-scale compression trial may be performed, using PALS-trained study collaborators as the compressors, to mimic more closely the actual forces generated when doing CPR. American Heart Association PALS CPR targets for rate and depth exist as ranges, given that humans are not able to identically compress with the exact same beat-to-beat rate and depth, as a device such as the testing setup 700 is designed to do. However, cardiac arrest outcomes are not changed by the subtle allowable differences within these range. This allowable compression variability is important to emulate to confirm the accuracy and precision of AR-CPR.

### Study Design, Procedure

The first tracking system 710 may be attached to the wrist of AHA PALS trained study collaborators. The first tracking system 710 and/or the AR-CPR IMU (e.g., sensors 210A-210C) may be activated to record compression rate and depth. The collaborator may subsequently perform two minutes of chest compressions on the manikin 730. Feedback may be withheld to ensure variability in the beat-to-beat compressions to test AR-CPRs function under the most variable conditions possible. Collaborators may perform 2 minutes of CPR. This interval may be repeated between two collaborators, resulting in 18 minutes of chest compression data, over which the system 200 may obtain 3240 rate, depth, and recoil measurements with AR-CPR and the first tracking system 710 set to record 30 datapoints per second. The AHA PALS guidelines demand 100-120 chest compressions per minute, or 1.7-2 compressions per second. As such, the data may be averaged over two seconds to directly compare the average measured rate (CPM) and depth (cm) for AR-CPR and the first tracking system 710.

### Outcome Measures, Statistical Analysis, and Hypothesis

The AR-CPR system 200 may continuously record the rate and depth of compressions, to directly compare them for equivalence to the first tracking system 710. For rate and depth, the primary outcome measures may be average compression rate (CPM) and depth (cm) over two second epochs as measured by AR-CPR system 200 and the first tracking system 710. For recoil, the primary outcome measures may be a percentage of recoil per compression, as calculated by the AR-CPR system 200 and/or the first tracking system 710. The equivalence of the AR-CPR measured rate, depth, and recoil measurement may be compared to that of the first tracking system 710, with an ICC of 0.95 being considered acceptable correlation. Additionally, the means of the 2 second epochs for AR-CPR system 200 and the first tracking system 710 may be compared using an unpaired t-test. The recorded rate, depth, and/or recoil measurements by AR-CPR system 200 may be substantially equivalent to those measured by the first tracking system 710. If non-equivalence is detected between the two systems 200, 710, the AR-CPR system 200 may be recalibrated and re-tested using this methodology until they are substantially identical.

### Alternatives

The manikin 730 may have built in functionality that can measure rate, depth, and/or recoil in real-time. Utilizing this data as the validated standard may allow the system 200 and method 300 to determine AR-CPRs rate, depth, and/or recoil accuracy and precision.

Although the present disclosure has been described in connection with preferred embodiments thereof, it will be appreciated by those skilled in the art that additions, deletions, modifications, and substitutions not specifically described may be made without departing from the spirit and scope of the disclosure as defined in the appended claims.

1. A system for facilitating cardiopulmonary resuscitation (CPR) by a CPR performer on a patient, the system comprising:
a first sensor configured to be positioned at least partially between a hand of the CPR performer and a chest of the patient, wherein the first sensor is configured to measure a depth of compressions performed by the CPR performer on the patient;
a second sensor configured to be positioned at least partially between the hand of the CPR performer and the chest of the patient, wherein the second sensor is configured to measure a rate of the compressions; and
a third sensor configured to be positioned at least partially between the hand of the CPR performer and the chest of the patient, wherein the third sensor is configured to measure a recoil of the compressions; and
a computing system configured to:
receive data from the first sensor, the second sensor, and the third sensor;
compare the data to stored data in a library that corresponds to the data received from the first sensor, the second sensor, and the third sensor; and
generate one or more outputs in response to the comparison.

2. The system of claim 1, wherein the stored data comprises pediatric CPR guidelines, and wherein the one or more outputs instruct the CPR performer how to modify the CPR on the patient to reduce differences between the received data and the stored data.

3. The system of claim 1, further comprising a display configured to display the one or more outputs.

4. The system of claim 3, wherein the display comprises an augmented reality (AR) headset configured to be positioned on a head of the CPR performer.

5. The system of claim 3, wherein the one or more outputs comprise:
a measured depth indicator corresponding to the depth of the compressions measured by the first sensor; and
a measured rate indicator corresponding to the rate of the compressions measured by the first sensor.

6. The system of claim 5, wherein the one or more outputs also comprise:
a predetermined depth range based upon the stored data; and
a predetermined rate range based upon the stored data.

7. The system of claim 6, wherein the predetermined depth range and the predetermined rate range form a box, wherein the measured depth indicator and the measured rate indicator being inside the box indicate that the CPR performed by the CPR performer is satisfactory, and wherein one or both of the measured depth indicator and the measured rate indicator being outside the box indicate that the CPR performed by the CPR performer is unsatisfactory and should be modified to move the measured depth indicator and the measured rate indicator inside the box.

8. The system of claim 1, wherein the one or more outputs are transmitted back to, and cause vibration within, the first sensor, the second sensor, the third sensor, or a combination thereof.

9. The system of claim 8, wherein:
the CPR performer is instructed to modify the depth of the compressions based at least partially upon a strength of the vibrations, and
the CPR performer is instructed to modify the rate of the compressions based at least partially upon a rate of the vibrations.

10. The system of claim 1, further comprising a fourth sensor configured to be positioned at least partially between the hand of the CPR performer and the chest of the patient, wherein the fourth sensor is configured to measure a location of an internal member of the patient, and wherein the one or more outputs instruct the CPR performer to move the hand with respect to the internal member.

11. A system for facilitating cardiopulmonary resuscitation (CPR) by a CPR performer on a patient, the system comprising:
a plurality of sensors comprising:
a first sensor configured to be positioned at least partially between a hand of the CPR performer and a chest of the patient, wherein the first sensor is configured to measure a depth of compressions;
a second sensor configured to be positioned at least partially between the hand of the CPR performer and the chest of the patient, wherein the second sensor is configured to measure a rate of compressions;
a third sensor configured to be positioned at least partially between the hand of the CPR performer and the chest of the patient, wherein the third sensor is configured to measure a recoil of compressions;
a fourth sensor configured to be positioned at least partially between the hand of the CPR performer and the chest of the patient, wherein the fourth sensor is configured to measure a location of a heart of the patient;
a fifth sensor configured to be positioned proximate to a mouth of the patient, wherein the fifth sensor is configured to measure a level of carbon dioxide that is released at an end of an exhaled breath;
a sixth sensor positioned at least partially around or on the patient, wherein the sixth sensor is configured to measure a blood pressure of the patient;
a seventh sensor positioned at least partially within or in contact with an artery, an arteriole, or a capillary of the patient, wherein the seventh sensor is configured to measure an oxygen saturation of the patient;
an eighth sensor positioned at least partially within an artery of the patient, wherein the eighth sensor is configured to measure an arterial pressure of the patient; and
a ninth sensor positioned on a head of the patient, an abdomen of the patient, or both, wherein the ninth sensor is configured to measure an oxygenation of tissues;
a computing system configured to:
receive data from the sensors;
compare the data to a library, wherein the library comprises stored data that corresponds to the data received from one or more of the sensors, and wherein the stored data comprises pediatric CPR guidelines provided by the American Heart Association (AHA); and
generate one or more outputs in response to the comparison to instruct the CPR performer how to modify the CPR on the patient to reduce differences between the received data and the stored data; and
an augmented reality (AR) headset configured to be positioned on a head of the CPR performer and to display the one or more outputs, wherein the one or more outputs comprise:
a measured depth indicator corresponding to the depth of the compressions measured by the first sensor;
a measured rate indicator corresponding to the rate of the compressions measured by the second sensor;
a predetermined depth range based upon the stored data; and
a predetermined rate range based upon the stored data, wherein the predetermined depth range and the predetermined rate range form a box, wherein the measured depth indicator and the measured rate indicator being inside the box indicate that the CPR performed by the CPR performer is satisfactory, and wherein one or both of the measured depth indicator and the measured rate indicator being outside the box indicate that the CPR performed by the CPR performer is unsatisfactory.

12. The system of claim 11, wherein the first sensor, the second sensor, the third sensor, or a combination thereof comprises an accelerometer, a gyroscope, and a geomagnetic sensor.

13. The system of claim 11, further comprising a defibrillator positioned at least partially between the hand of the CPR performer and the chest of the patient, wherein the defibrillator comprises the first sensor, the second sensor, the third sensor, the fourth sensor, or a combination thereof.

14. The system of claim 11, wherein one or both of the measured depth indicator and the measured rate indicator being outside the box indicate that the CPR performed by the CPR performer should be modified to move the measured depth indicator and the measured rate indicator inside the box.

15. The system of claim 11, wherein the one or more outputs also comprise a command to instruct the CPR performer to increase the depth of the compressions, decrease the depth of the compressions, increase the rate of the compressions, decrease the rate of the compressions, or a combination thereof.

16. A method for facilitating cardiopulmonary resuscitation (CPR) by a CPR performer on a patient, the method comprising:
measuring a depth of compressions, a rate of the compressions, and a recoil of the compressions performed by the CPR performer on the patient using one or more sensors that are positioned at least partially between a hand of the CPR performer and a chest of the patient;
comparing the measured depth, rate, and recoil to a stored depth, rate, and recoil that are stored in a library; and
generating one or more outputs in response to the comparison.

17. The method of claim 16, further comprising displaying the one or more outputs on an augmented reality (AR) headset that is configured to be positioned on a head of the CPR performer.

18. The method of claim 16, wherein the one or more outputs comprise:
a measured depth indicator corresponding to the depth of the compressions measured by the one or more sensors;
a predetermined depth range based upon the stored depth;
a measured rate indicator corresponding to the rate of the compressions measured by the one or more sensors;
a predetermined rate range based upon the stored rate;
a measured recoil indicator corresponding to the recoil of the compressions measured by the one or more sensors; and
a predetermined recoil range based upon the stored recoil.

19. The method of claim 18, wherein the predetermined depth range and the predetermined rate range form a box, and wherein a location of the measured depth indicator and the measured rate indicator with respect to the box instructs the CPR performer how to modify the compressions.

20. The method of claim 16, wherein the one or more outputs instruct the CPR performer to modify the compressions in response to the recoil being less than 95%.
